(19) United States
(12) Patent Application Publication
(10) Pub. No.: US 2010/0010029 A1
(43) Pub. Date: Jan. 14, 2010

(54) Acute Pain Medications Based on Fast Acting Diclofenac-Opioid Combinations

(75) Inventors: William R. Maichle, Lowell, NC (US); Carl L. Whatley, Montgomery, AL (US); Giorgio Reiner, Como (IT); Alberto Reiner, Como (IT)

Correspondence Address: PATENT CORRESPONDENCE;ARNALL GOLDEN GREGORY LLP, 171 17TH STREET NW, SUITE 2100, ATLANTA, GA, 30363, US

(73) Assignees: Kowa Phamaceuticals America, Inc., Montgomery, AL (US); APR Applied Pharma Research, S.A., Balerna (CH)

(21) Appl. No.: 12/298,922

(22) PCT Filed: May 2, 2007
(86) PCT No.: PCT/US2007/010717

§ 371 Date: May 5, 2009

Related U.S. Application Data
(60) Provisional application No. 60/797,088, filed on May 3, 2006.

(51) Int. Cl. A61K 31/4355; A61K 31/445; A61K 31/34; A61K 31/196

(52) U.S. Cl. 514/282; 514/315; 514/468; 514/567

(57) ABSTRACT

Provided are combined oral dosage forms for the treatment of pain, particularly combined dosage forms that are specially formulated for rapid bioavailability, and that contain diclofenac potassium and an opioid selected from hydrocodone, oxycodone, fentanyl and tramadol.

### RELATED APPLICATIONS

The present application claims priority under 35 U.S.C. § 119 to U.S. Ser. No. 60/797,088, filed May 3, 2006.

### FIELD OF THE INVENTION

The present invention relates to combined oral dosage forms for the treatment of pain. In particular, the invention relates to combined dosage forms that are specially formulated for rapid bioavailability, and that contain diclofenac potassium and an opioid that is preferably selected from hydrocodone, oxycodone, fentanyl and tramadol.

### BACKGROUND OF THE INVENTION

Numerous drugs are marketed for the treatment of pain, including non-steroidal anti-inflammatory drugs (NSAIDs) and opioids. NSAIDs that are often prescribed for the treatment of acute pain include, for example, acetaminophen, ibuprofen, naproxen, diclofenac, ketoprofen, nabumetone and salicyclic acid. Opioids that are often prescribed for the treatment of moderate to severe acute pain include, for example, morphine, hydromorphone, codeine, hydrocodone, oxycodone, tramadol and fentanyl.

Recently, several fixed combination dosage forms of NSAIDs and opioids have been developed and marketed for the treatment of pain, including Vicodin®, Vicoprofen®, Percocet® and Ultracet®. These drugs contain combinations of acetominophen and hydrocodone bitartrate in doses of 500 mg/5 mg, 750 mg/7.5 mg, and 660 mg/10 mg (Vicodin®), ibuprofen and hydrocodone bitartrate in a dose of 200 mg/7.5 mg (Vicoprofen®), acetaminophen and oxycodone hydrochloride in doses of 325 mg/10 mg, 325 mg/2.5 mg, 325 mg/5 mg, 325 mg/7.5 mg, 500 mg/7.5 mg, and 650 mg/10 mg (Percocet®), and acetaminophen and tramadol hydrochloride in a dose of 325 mg/37.5 mg (Ultracet®). Users of these drugs are typically instructed to take one or two pills as needed for pain, four to six times daily, with the total dosage not to exceed a certain number of pills per day.

Onset and duration of action are two parameters that are often used to evaluate the clinical efficacy and utility of an acute pain medication. Pharmacokinetic parameters used to predict onset and duration of action include Cₘₐₓ (i.e the maximum concentration of the drug in blood), and tₘₐₓ (i.e. the time to reach Cₘₐₓ). The coefficient of variation (“CV”) for these variables is also often taken into account, because it measures the consistency of the pharmacokinetic profile for the drug, which in turn predicts the consistency of onset and duration. Some drugs, such as extended release oxycodone formulations (commercially marketed as Oxycontin®), are formulated to provide quick onset and prolonged duration, by releasing a portion of the active ingredient almost immediately upon ingestion, and another portion over a prolonged period of time. Other drugs are formulated for either rapid response or delayed response, by hastening or delaying the bioavailability of the active agent in the drug.

### SUMMARY OF THE INVENTION

The present invention provides rapidly bioavailable oral dosage forms, containing diclofenac and an opioid selected from hydrocodone, oxycodone, tramadol or fentanyl, that have a high Cₘₐₓ and a short tₘₐₓ. The dosage forms of the present invention provide numerous advantages over either ingredient alone, or combinations of the ingredients in conventional immediate release dosage forms, including:
a significant reduction in the time to onset of action;
a significant improvement in the level of pain reduction initially observed on the VAS scale;
reduced requirements for diclofenac or opioid dosing, when compared to either ingredient alone, or traditional immediate release formats;
pain relief over extended periods of time, up to eight hours after administration, that is comparable to conventional dosage forms that provide longer pharmacokinetics; and
significant reductions in the coefficients of variation for Cₘₐₓ and tₘₐₓ when compared to conventional dosage forms.

Therefore, in one embodiment, the invention provides a process for treating pain in a mammal which comprises administering to the mammal an amount of a pharmaceutical composition effective to provide an analgesic effect, said pharmaceutical composition comprising diclofenac or a pharmaceutically acceptable salt thereof and an opioid that is preferably selected from hydrocodone, oxycodone, tramadol or fentanyl, or a pharmaceutically acceptable salt thereof, wherein: (a) said composition exhibits a tₘₐₓ for said diclofenac of from about 10 minutes to about 30 minutes; and (b) the weight ratio of diclofenac to opioid is within a range that the administration of a therapeutic amount of said composition to a mammal will provide a greater analgesic effect than the effect obtainable by use of either said diclofenac or said opioid alone. In another embodiment the invention provides a pharmaceutical composition which comprises diclofenac or a pharmaceutically acceptable salt thereof and an opioid that is preferably selected from hydrocodone, oxycodone, tramadol or fentanyl, or a pharmaceutically acceptable salt thereof, wherein: (a) said composition exhibits a tₘₐₓ for said diclofenac of from about 10 minutes to about 30 minutes; and (b) the weight ratio of diclofenac to opioid is within a range that the administration of a therapeutic amount of said composition to a mammal will provide a greater analgesic effect than the effect obtainable by use of either said diclofenac or said opioid alone.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE FIGURES

FIG. 1 compares the pharmacokinetic profile of a 50 mg: rapidly bioavailable tablet of diclofenac potassium (PRO-571), overlaid against the pharmacokinetic profile of Cataflam®.

FIG. 2 contains a graphical summary of headache intensities during the first twenty four hours after treatment, comparing a fast release diclofenac sachet formulation and placebo, as described in Example 3.

### DETAILED DESCRIPTION OF THE INVENTION

### Definitions

As used in the specification and claims, the singular forms a, an and the include plural references unless the context clearly dictates otherwise. For example, the term a pharmaceutical excipient may refer to one or more pharmaceutical excipients for use in the presently disclosed formulations and methods.

USP means the United States Pharmacopoeia and National Formulary (USP 28-NF 23). Rockville, Md.: United States Pharmacopoeia Convention; 2004, unless stated to the contrary. USP 28 <701> refers to physical test 701, disintegration, contained on pages 2411-2412 of the USP. USP 28 <711> refers to physical test 711, dissolution, contained on pages 2412-2414 of the USP. The particular test employed will vary from drug to drug, but measurements for diclofenac and other drugs that precipitate under acidic conditions are preferably made in phosphate buffered water at pH=6.8 and 37° C. Other acid-soluble drugs can typically be measured in water buffered by 0.1M HCl at 37° C. The drugs may generally be tested at 50 RPM using a USP Type II dissolution apparatus, in 500 or 900 ml of solution, at 37° C.

Alternatively, tramadol may be tested in a USP type I basket at 100 rpm in 0.1N HCl; oxycodone may be tested in a USP type I basket at 100 rpm in a phosphate buffered pH 7.2 aqueous medium; hydrocodone may be tested in a USP type II paddle device at 50 rpm in a phosphate buffered pH 7.2 aqueous medium; dihydrocodeine may be tested in a USP type I basket at 100 rpm in water.

A dosage form, as used herein, refers to a formulation that is ready for administration to a subject. As used herein, it may refer to solid dosage forms, including, but not limited to, tablets, powders and capsules, tablets being the most preferred. Alternatively, it may refer to a liquid dosage form such as a solution or a suspension. An “intact” dosage form refers to a dosage form which is ingested in the form it is provided. Intact dosage forms are therefore to be distinguished from orally disintegrating tablets which disintegrate in the mouth before being ingested or effervescent tablets which are dissolved in water before being ingested. In preferred embodiments of this invention, the dosage form is a tablet, and the tablets are ingested in an intact form.

When doses are given for a drug and its salt, it will be understood that the calculated dose is based on the molecular weight of the active pharmaceutical ingredient, which includes the cationic and anionic species in the case of a salt, and just the base when the active principle is not present as a salt.

When ranges are given by specifying the lower end of a range separately from the upper end of the range, it will be understood that the range can be defined by selectively combining any one of the lower end variables with any one of the upper end variables that is mathematically possible.

When used herein the term “about” will compensate for variability allowed for in the pharmaceutical industry and inherent in pharmaceutical products, such as differences in product strength due to manufacturing variation and time-induced product degradation. When a strength is recited, the term allows for any variation which in the practice of pharmaceuticals would allow the product being evaluated to be considered bioequivalent to the recited strength.

When clinical or biopharmaceutic results are reported, it will be understood that the results are preferably obtained to a statistically significant level, which in alternative embodiments is defined as p<1.0, p<0.1, or p<0.05.

### Discussion

In a first principal embodiment the invention provides a pharmaceutical composition which comprises diclofenac or a pharmaceutically acceptable salt thereof and an opioid that is preferably selected from hydrocodone, oxycodone, tramadol or fentanyl, or a pharmaceutically acceptable salt thereof, wherein: (a) said composition exhibits a tₘₐₓ for said diclofenac of from about 10 minutes to about 30 minutes; and (b) the weight ratio of diclofenac to opioid is within a range that the administration of a therapeutic amount of said composition to a mammal will provide a greater analgesic effect than the effect obtainable by use of either said diclofenac or said opioid alone. In a second principal embodiment the invention provides a process for treating pain in a mammal which comprises administering to the mammal an amount of the pharmaceutical composition of the present invention effective to provide an analgesic effect. The dosing for the combined dosage forms of the present invention preferably provides relief form up to or greater than 4, 6 or eight hours, so that the dosage forms can be dosed twice daily, three times daily, four times daily, or as needed not to exceed four to six administrations per day, but in a particularly preferred embodiment, the dosing is three times daily (i.e. t.i.d.).

Diclofenac is chemically described as [(2,6-dichloro-anilino)-2-phenyl]-2-acetic acid. The potassium salt of the molecule is represented by the following chemical structure:

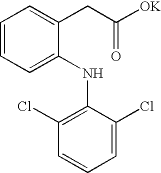

For purposes of this invention, diclofenac can be administered as the acid form or as any pharmaceutically acceptable salt that demonstrates adequate stability upon storage and bioavailability upon administration, but is preferably administered as diclofenac sodium or diclofenac potassium.

The dosage form preferably comprises from about 10 mg. to about 100 mg., more preferably from about 15 mg. to about 60 mg., and most preferably from about 25 mg. to about 50 mg., or about 25 mg. or about 50 mg. specifically, of diclofenac potassium or diclofenac base (as diclofenac potassium, diclofenac sodium or diclofenac acid). In addition, the dosage form preferably meets one or more of the following pharmacokinetic criteria for the diclofenac:
a tₘₐₓ of from about 5 or 10 to about 40, 35, 30, 25 or 20 minutes, most preferably from about 10 to about 20 minutes (preferably when tested in a fasted state);
an inter-subject coefficient of variability for said tₘₐₓ of preferably less than about 80, 75, 60, 50, 45, 40, 35, 30% or 25%;
a Cₘₐₓ of from about 1200, 1300, 1400, 1500 or 1600 to about 2500 ng/ml for a 50 mg. dose of diclofenac potassium or diclofenac (i.e. 0.026 ml⁻¹ to about 0.05 ml⁻¹ when normalized), preferably from about 1300 to about 2500 ng/ml for a 50 mg. dose (i.e. from about 0.026 liter⁻¹ to about 0.05 liter⁻¹ when normalized) and more preferably from about 1500 to about 2500 ng/ml for a 50 mg. dose (i.e. from about 0.03 liter⁻¹ to about 0.05 liter⁻¹ when normalized) (preferably when tested in a fasted state);
an inter-subject coefficient of variability for said Cₘₐₓ of less than about 70, 60, 50, 45 or 40%;
a single plasma concentration peak reflecting predominant absorption in the upper portion of the gastrointestinal tract; and/or
a disintegration or dissolution time of less than about 20, 15, 10, 5 or 3 minutes when tested according to USP 28 <701> or USP 28 <711> (Q=85%).

The dosage form also comprises an opioid that is preferably selected from hydrocodone, oxycodone, tramadol or fentanyl, or a pharmaceutically acceptable salt thereof, in a therapeutically effective amount. These opioid ingredients may be mixed intimately with the diclofenac so that they are released from the dosage form at approximately the same rate, or they may be specially formulated for release distinct from the NSAID, as in a bilayer tablet (as discussed below). Alternatively, the opioid may be coated with a suitable protective agent such as a methacrylic copolymer, for protection from the alkaline effects of the bicarbonate buffer.

Hydrocodone bitartrate is an opioid analgesic and antitussive and occurs as fine, white crystals or as a crystalline powder. The chemical name is 4,5α-Epoxy-3-methoxy-17-methylmorphinan-6-one tartrate (1:1) hydrate (2:5). It has the following structural formula:

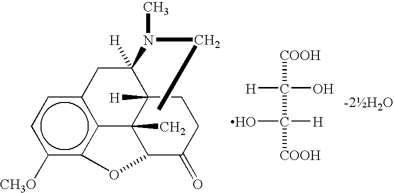

Preferred weight ratios of diclofenac (or a pharmaceutically acceptable salt thereof) to hydrocodone (or a pharmaceutically acceptable salt thereof) range from about 1.25:1 to about 20:1, and more preferably range from about 2.5:1 to about 10:1. Specific diclofenac K/hydrocodone bitartrate formulations with which the invention can be practiced are set forth in Table 1 below, though it will be understood that similar formulations could be prepared using the same weight of diclofenac or hydrocodone base or another pharmaceutically acceptable salt thereof:

TABLE 1

| | | Hydrocodone Bitartrate |
|---|---|---|
| Formulation | Diclofenac K (mg.) | (mg.) |
| 1 | 12.5 | 2.5 |
| 2 | 12.5 | 5.0 |
| 3 | 12.5 | 7.5 |
| 4 | 12.5 | 10.0 |
| 5 | 25.0 | 2.5 |
| 6 | 25.0 | 5.0 |
| 7 | 25.0 | 7.5 |
| 8 | 25.0 | 10.0 |
| 9 | 37.5 | 2.5 |
| 10 | 37.5 | 5.0 |
| 11 | 37.5 | 7.5 |
| 12 | 37.5 | 10.0 |
| 13 | 50.0 | 2.5 |
| 14 | 50.0 | 5.0 |
| 15 | 50.0 | 7.5 |
| 16 | 50.0 | 10.0 |

Oxycodone is a semisynthetic narcotic derived from the opium alkaloid, thebain, with multiple actions qualitatively similar to morphine. It has the following chemical name: 14-hydroxydihydrocodeinone. The hydrochloride salt of oxycodone may be represented by the following structural formula:

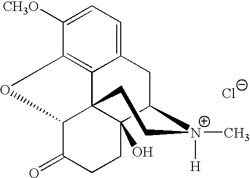

Preferred weight ratios of diclofenac (or a pharmaceutically acceptable salt thereof) to oxycodone (or a pharmaceutically acceptable salt thereof) range from about 1.25:1 to about 20:1, and more preferably range from about 2.5:1 to about 10:1. Specific diclofenac K/oxycodone HCl formulations with which the invention can be practiced are set forth in Table 2 below, though it will be understood that similar formulations could be prepared, using the same weight of diclofenac or oxycodone base or another pharmaceutically acceptable salt thereof:

TABLE 2

| Formulation | Diclofenac K (mg.) | Oxycodone HCl (mg.) |
|---|---|---|
| 1 | 12.5 | 2.5 |
| 2 | 12.5 | 5.0 |
| 3 | 12.5 | 7.5 |
| 4 | 12.5 | 10.0 |
| 5 | 25.0 | 2.5 |
| 6 | 25.0 | 5.0 |
| 7 | 25.0 | 7.5 |
| 8 | 25.0 | 10.0 |
| 9 | 37.5 | 2.5 |
| 10 | 37.5 | 5.0 |
| 11 | 37.5 | 7.5 |
| 12 | 37.5 | 10.0 |
| 13 | 50.0 | 2.5 |
| 14 | 50.0 | 5.0 |
| 15 | 50.0 | 7.5 |
| 16 | 50.0 | 10.0 |

Tramadol is a centrally acting synthetic opioid analgesic. The chemical name for tramadol hydrochloride is (±)cis-2-9[(dimethylamino)methyl]-1-(3-methoxyphenyl) cyclohexanol hydrochloride. Its structural formula is:

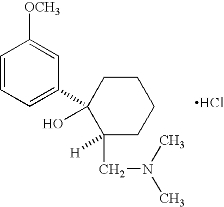

Preferred weight ratios of diclofenac (or a pharmaceutically acceptable salt thereof) to tramadol (or a pharmaceutically acceptable salt thereof) range from about 0.25:1 to about 2:1, and more preferably range from about 0.5:1 to about 1:1. Specific diclofenac K/tramadol HCl formulations with which the invention can be practiced are set forth in Table 3 below, although it will be understood that similar formulations could be prepared, using the same weight of diclofenac or tramadol base or another pharmaceutically acceptable salt thereof:

TABLE 3

| Formulation | Diclofenac K (mg.) | Tramadol HCl (mg.) |
|---|---|---|
| 1 | 12.5 | 25.0 |
| 2 | 12.5 | 37.5 |
| 3 | 12.5 | 50.0 |
| 4 | 25.0 | 25.0 |
| 5 | 25.0 | 37.5 |
| 6 | 25.0 | 50.0 |
| 7 | 37.5 | 25.0 |
| 8 | 37.5 | 37.5 |
| 9 | 37.5 | 50.0 |
| 10 | 50.0 | 25.0 |
| 11 | 50.0 | 37.5 |
| 12 | 50.0 | 50.0 |

Fentanyl is a potent synthetic opioid analgesic having the chemical name N-Phenyl-N-(1-(2-phenylethyl)-4-piperidinyl) propanamide. The chemical structure of the citrate salt of fentanyl is depicted below:

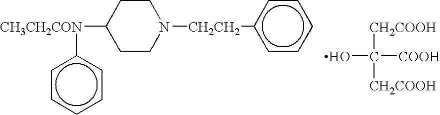

Preferred weight ratios of diclofenac (or a pharmaceutically acceptable salt thereof) to fentanyl (or a pharmaceutically acceptable salt thereof) range from about 15:1 to about 250:1, and more preferably range from about 50:1 to about 225:1, and still more preferably from about 100:1 to about 200:1. Specific diclofenac K/fentanyl citrate formulations with which the invention can be practiced are set forth in Table 4 below, although it will be understood that similar formulations could be prepared, using the same weight of diclofenac or fentanyl base or another pharmaceutically acceptable salt thereof:

TABLE 4

| | Diclofenac K | Fentanyl Citrate |
|---|---|---|
| Form. | (mg.) | (mg.) |
| 1 | 12.5 | 0.2 |
| 2 | 12.5 | 0.4 |
| 3 | 12.5 | 0.6 |
| 4 | 12.5 | 0.8 |
| 5 | 12.5 | 1.0 |
| 6 | 12.5 | 1.2 |
| 7 | 12.5 | 1.4 |
| 8 | 12.5 | 1.6 |
| 9 | 25.0 | 0.2 |
| 10 | 25.0 | 0.4 |
| 11 | 25.0 | 0.6 |
| 12 | 25.0 | 0.8 |
| 13 | 25.0 | 1.0 |
| 14 | 25.0 | 1.2 |
| 15 | 25.0 | 1.4 |
| 16 | 25.0 | 1.6 |
| 17 | 37.5 | 0.2 |
| 18 | 37.5 | 0.4 |
| 19 | 37.5 | 0.6 |
| 20 | 37.5 | 0.8 |
| 21 | 37.5 | 1.0 |
| 22 | 37.5 | 1.2 |
| 23 | 37.5 | 1.4 |
| 24 | 37.5 | 1.6 |
| 25 | 50.0 | 0.2 |
| 26 | 50.0 | 0.4 |
| 27 | 50.0 | 0.6 |
| 28 | 50.0 | 0.8 |
| 29 | 50.0 | 1.0 |
| 30 | 50.0 | 1.2 |
| 31 | 50.0 | 1.4 |
| 32 | 50.0 | 1.6 |

Other opioids with which the invention can be practiced include: morphine (preferably 10-100 mg. or 30-60 mg.), hydromorphone (preferably 1-15 mg. or 5-10 mg.), methadone preferably 5-40 mg. or 10-30 mg.), levorphanol (preferably 0.5-10 mg. or 2-8 mg.), or oxymorphone (preferably 2-25 or 5-20 mg.).

The combined dosage forms of the present invention may be evaluated in numerous acute pain models, including models based upon: (i) post-operative dental pain, (ii) pain after orthopedic skeletal surgery, (iii) flare-up in rheumatic conditions, (iv) pain after gynecological surgery, (v) post-episiotomy pain, (vi) dysmenorrheal, and (vii) ankle sprains. In one embodiment, the combined dosage form is not inferior when compared to each of the active ingredients administered individually against one or more or all of the following endpoints:
Pain intensity on VAS (i.e. average reduction in pain on a VAS scale (0 mm=no pain; 100 mm=unbearable pain) measured at various time endpoints, such as 15 minutes, 30 minutes, 45 minutes, 60 minutes, 90 minutes, 2 hours, 4 hours, 6 hours, 8 hours, 12 hours and 24 hours). In a preferred embodiment, the average pain reduction from baseline within two hours is greater than 20, 25, 30, 35, 40, 45 or 50 mm on the VAS scale.
Pain intensity on 4 point verbal scale (i.e. none, mild, moderate, or severe), measured at various time endpoints, such as 15 minutes, 30 minutes, 45 minutes, 60 minutes, 90 minutes, 2 hours, 4 hours, 6 hours, 8 hours, 12 hours and 24 hours)
Time to onset of analgesic effect, such as 15 minutes, 30 minutes, 45 minutes, 60 minutes, 90 minutes, 2 hours, 4 hours, 6 hours, 8 hours, 12 hours and 24 hours as measured by statistically significant differences in VAS, or by statistically significant instances of no pain;
Time to perceptible/meaningful pain relief (i.e. time to reach perceptible or meaningful pain relief, measured as “a little,” “moderate,” “a lot,” or “complete”)
Patient's global evaluation (very poor, poor, no opinion, good, very good)

In an even more preferred embodiment, the combination dosage form is superior to each of the active ingredients administered individually when measured against one or more of the foregoing migraine or acute pain endpoints, and not inferior when measured against one or more or the remainder of the endpoints. In any of the models or clinical comparisons, the comparator active ingredients may be formulated to give the same pharmacokinetic profile as in the combined dosage form, or they may simply constitute immediate release formulations that meet conventional pharmacokinetic profiles, as defined for many drugs in the United States Pharmacopoeia, or as defined below in greater detail.

The dosage forms of the present invention can take various forms, including oral solutions, oral suspensions, powders for oral suspension, tablets, capsules (e.g, hard and soft gelatin capsules), mucoadhesive films, and orally dissolving tablets, among others. The compositions of the present invention may be prepared by bringing the active ingredients into association with (e.g., by mixing with) the pharmaceutically acceptable carrier. Any suitable carrier known in the art can be used to prepare the pharmaceutical composition of the present invention. A solid carrier can be, for example, one or more substances which may also act as flavoring agents, lubricants, solubilisers, suspending agents, fillers, compression aids, binders or tablet-disintegrating agents; it can also be an encapsulating material. In powders the carrier is a finely divided solid which is in admixture with the finely divided active ingredient. In tablets the active ingredient is mixed with a carrier having the necessary compression properties in suitable proportions and compacted in the shape and size desired. The powders and tablets preferably contain up to 99%, e.g., from 0.03 to 99%, preferably 1 to 80% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid form compositions include, for example, solutions, suspensions, emulsions, syrups, elixirs and pressurized compositions. The active ingredient, for example, can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilisers, emulsifiers, buffers, preservatives, sweeteners, flavoring agents, suspending agents, thickening agents, colors, viscosity regulators, stabilizers or osmoregulators. Suitable examples of liquid carriers for oral administration include water (particularly containing additives as above, e.g., cellulose derivatives, preferably sodium carboxymethyl cellulose solution), alcohols (including monohydric alcohols (including monohydric alcohols and polyhydric alcohols, e.g., glycerine and non-toxic glycols) and their derivatives, and oils (e.g., fractionated coconut oil and arachis oil). Preferably the compositions of the present invention are administered orally either in liquid or solid composition form.

The active ingredients can be in one unitary formulation, so that each is released at the same rate when dissolved in the stomach. The unitary formulation can be a conventional immediate release formulation, or a fast release formulation. For purposes of this disclosure, the term “fast release” is defined to mean that the dosage form yields a dissolution or disintegration time of less than about 30, 20, 15, 10, 5 or 3 minutes when tested according to USP 28 <701> or USP 28 <711> (Q=85%). The term “immediate release” is defined to mean that the dosage form yields a dissolution or disintegration time of less than about 90, 60 or 45 minutes (preferably 60 minutes) (85% or more dissolving or disintegrating), and typically greater than about 5, 10, 15 or 20 minutes (preferably 20 minutes) (35% or less dissolving or disintegrating), when tested according to USP 28 <701> or USP 28 <711>.

Of course, it is also possible, and in most instances preferable, to formulate the active ingredients in two separate vehicles, so that differing pharmacokinetic profiles are observed for the diclofenac and the opioid. This can be done, for example, in the form of a bilayer tablet, that contains a “fast release” layer containing the diclofenac, and an “immediate release” layer that contains the opioid (measured as defined above for the terms “fast release” and “immediate release.” The layers could be compressed one against the other, so that each is exposed immediately to biological fluids upon ingestion, or one could form the outer layer of a shell that must dissolve completely before exposing the inner/second layer to the biological fluids. Alternatively, separate beads that have different release profiles could be constructed for containing the diclofenac and the opioid, and proportionate amounts of the beads could be added to a hard gelatin capsule in the preparation of a capsule dosage form.

Therefore, in one embodiment the diclofenac potassium and opioid achieve fast release when tested according to USP 28 <701> or USP 28 <711> (Q=85%). In another embodiment the diclofenac potassium and opioid achieve immediate release when tested according to USP 28 <701> or USP 28 <711> (Q=85%). In yet another embodiment the diclofenac potassium achieves fast release, and the opioid achieves immediate release, when tested according to USP 28 <701> or USP 28 <711> (Q=85%).

The invention also contemplates the concomitant administration of diclofenac and an opioid in separate dosage forms. These separate dosage forms preferably employ the same dose amounts, and the same pharmacokinetics, as described above for each ingredient in a combination dosage form. Likewise, when combined in a concomitant administration regime, the separate dosage forms preferably meet the clinical endpoints described above. The invention further contemplates kits that comprise the separate dosage forms in a unitary package, with appropriate instructions for use.

For purposes of this invention, the term “concomitant administration” shall refer to “simultaneous administration” or “co-timely administration.” The term “co-timely” as to drug administration shall mean administration of a second drug for migraine relief while a first drug for migraine relief is present in a therapeutically effective amount. It is to be understood that in some instances this will require sequential administration. In some instances, multiple routes of administration will be employed such as intravenous or subcutaneous injection of an opioid, while diclofenac is taken orally from prior to or subsequent to such opioid injection.

Buffering agents are not critical to the invention, but are preferably used to provide a rapid rate of onset for the diclofenac. In a preferred embodiment, the buffering agent controls the pH of the portion of the formulation that contains diclofenac when dissolved in water, and preferably yields a pH greater than about 6.8, 7.0, 7.2, or 7.4, and less than about 7.8, 7.7 or 7.6, when mixed with 50 ml or 100 or 200 ml. of water at 25 degrees Celsius. Particularly preferred buffering agents are alkali metal carbonates and bicarbonates and these agents are preferably employed in a weight ratio relative to the diclofenac of greater than about 1:5, 2:5, 2:1, 3:1 or 5:1. If desired, an upper limit on the buffer:diclofenac ratio can be placed at about 20:1, 10:1, 5:1, 1:1, 4:5 or 3:5. Ranges can be selected from any two of the foregoing values that are mathematically possible. In a preferred embodiment, the buffer:diclofenac weight ratio ranges from about 1:5 to about 4:5. Particularly preferred alkali metal bicarbonates are sodium bicarbonate and potassium bicarbonate.

### EXAMPLES

### Example 1

### Formulations

A suitable dose of opioid that is preferably selected from hydrocodone, oxycodone, fentanyl or tramadol can be combined with diclofenac potassium in a therapeutically effective amount (TE) to arrive at the following formulations:

Composition dissolving instantly in water

| Active ingredients | | |
|---|---|---|
| 1) Diclofenac potassium salt*: | 50 | mg |
| 2) Opioid | TE | |
| 3) Potassium bicarbonate: | 22 | mg |
| 4) Mint flavoring on maltodextrin (1:2000)**: | 60 | mg |
| 5) Aniseed flavoring on maltodextrin (1:1000)***: | 104 | mg |
| Excipients and adjuvants | | |
| 6) Saccharin: | 4 | mg |
| 7) Aspartame: | 10 | mg |
| 8) Mannitol: | 50 | mg |
| 9) Saccharose*** *q.s.: | 2 | g |

*If it is desired to prepare compositions based on diclofenac sodium salt, it is advantageous to use sodium bicarbonate in a quantity of approximately 38% by weight based on the weight of the diclofenac sodium salt present.
Sodium carbonate may also be added to the sodium bicarbonate, maintaining the following optimum proportions: 27% of sodium bicarbonate and 4-5% of sodium carbonate, always based on the amount by weight of diclofenac sodium salt present.
**The title of the pure mint essence, as obtained according to the Dean-Stark method, is of 18% by weight; the related amount is therefore in this case of 10.8 mg.
***The title of the pure anise essence, as obtained according to the Dean-Stark method, is of 14.5% by weight, the related amount is therefore in this case of 16 mg.
****The presence of saccharose is not strictly necessary; in its absence, a composition having a very limited granulate content is obtained which is perfectly 20 soluble in contact with water. In that case, nothing is changed from the point of view of tolerability in contact with the mucosa and. from the point of view of the palatability of the drinkable solution.
RD—Recommended Dose (see Tables 1-4)

### Preparation

Components 1, 2, 3, 6, 7 and 8 are mixed in a suitable mixer, and the mixture so obtained is wetted with 95% ethanol. Granulation is carried out with a 66 mm mesh and the granulate is preferably dried in current of air.

Components 4, 5 and 9, which have already been granulated using a mesh of the same granulometry, are then added and the whole is mixed. The mixture is then introduced into a metering machine for filling packets or similar containers.

Tablet for dissolving in the mouth

| | |
|---|---|
| Active ingredients | |
| 1) Diclofenac potassium salt*: | 50 mg |
| 2) Opioid | TE |
| 3) Potassium bicarbonate: | 35 mg |
| 4) Mint flavoring on maltodextrin** | 50 mg |
| (1:2000) + gum arabic (E 414): | |
| 5) Aniseed flavoring (1:1000) | 120 mg |
| on maltodextrin*** + silicon | |
| dioxide(E551): | |
| Excipients and adjuvants | |
| 6) Saccharin: | 50 mg |
| 7) Aspartame: | 12 mg |
| 8) Mannitol: | 20 mg |
| 9) Saccharose****: | 300 mg |

*to**** see Example 1

Two layered tablet (fast and slow release)

| Fast release layer | | |
|---|---|---|
| 1) Diclofenac potassium salt: | 15 | mg |
| 2) Potassium bicarbonate: | 30 | mg |
| 3) Lactose: | 13.2 | mg |
| 4) Maize starch (intragranular): | 6 | mg |
| 5) Methyl cellulose: | 0.12 | mg |
| 6) Sodium laurylsulfate: | 0.06 | mg |
| 7) Maize starch (extragranular): | 9 | mg |
| 8) Crospovidone: | 0.6 | mg |
| 9) Sodium carboxymethylstarch: | 1.5 | mg |
| 10) Magnesium stearate: | 2.7 | mg |
| 11) Colloidal silicon dioxide: | 0.6 | mg |
| Slow release layer | | |
| 1) Opioid | TE | |
| 2) Lactose: | 32.2 | mg |
| 3) Polyvinylpyrrolidone: | 1.16 | mg |
| 4) Hydroxypropylmethylcellulose: | 70 | mg |
| 5) Magnesium stearate: | 0.84 | mg |
| 6) Colloidal silicon dioxide: | 0.21 | mg |
| 7) Talc: | 3.92 | mg |
| 9) Polyethylene glycol: | 0.56 | mg |

Tablet

| | | |
|---|---|---|
| 1) | Diclofenac potassium salt: | 50 mg |
| 2) | Opioid | TE |
| 3) | Mannitol: | 50 mg |
| 4) | Potassium bicarbonate: | 22 mg |
| 5) | Maize starch (intragranular): | 10 mg |
| 6) | Methyl cellulose: | 0.2 mg |
| 7) | Sodium laurylsulfate: | 0.1 mg |
| 8) | Maize starch (extragranular): | 15 mg |
| 9) | Crospovidone: | 1.0 mg |
| 10) | Sodium carboxymethylstarch: | 2.5 mg |
| 11) | Magnesium stearate: | 4.5 mg |
| 12) | Colloidal silicon dioxide: | 10 mg |

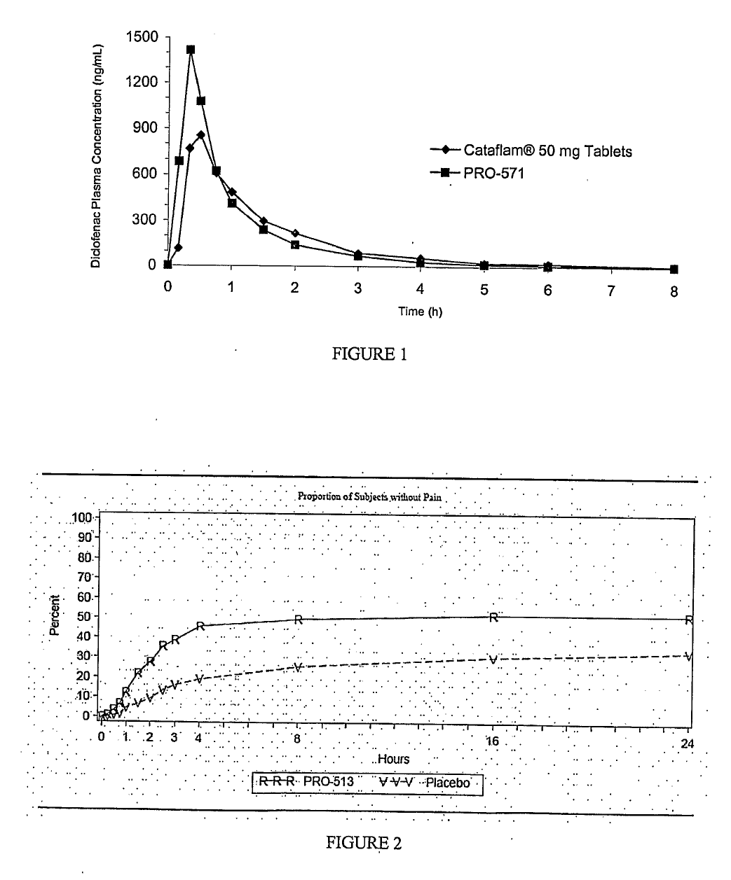

FIG. 1 compares the pharmacokinetic profile of the 50 mg. tablet of diclofenac potassium (PRO-571), overlaid against the pharmacokinetic profile of Cataflam®.

### Example 2

### Comparative Efficacy of Diclofenac Powder Against Migraine Headache

A randomized, double-blind, double-dummy multi-center, single dose, placebo- and active-controlled crossover study, with an eight hour evaluation was undertaken in adult migraine patients. 328 migraine patients with or without aura according to HIS criteria were randomized among treatments and a comparison made among treatments with a 50 mg. diclofenac potassium sachet formulation prepared substantially as described in Example 1, and demonstrating a tₘₐₓ of about 14 minutes, a 50 mg. diclofenac potassium sugar coated tablet marketed commercially as Cataflam®, and demonstrating a tₘₐₓ of about 52 minutes, and placebo. Patients were randomized to treatment for three separate migraine attacks, each attack treated with a different study medication. Results are reported in Table 5.

TABLE 5
Pain on Verbal Scale

| | Diclofenac-K | Diclofenac-K | |
|---|---|---|---|
| Parameter | Sachet | Tablet | Placebo |
| Pain free at 2 hours | % of patients | % of patients | % of patients |
| ITT pop | 24.7% | 18.5% | 11.7% |
| PP pop | 23.6% | 17.8% | 12.9% |
| Mod-sev | 24.2% | 17.0% | 12.5% |
| baseline pain | | | |
| Headache response 2 | 46.0% | 41.6% | 24.1% |
| hours* | | | |
| Sustained response | 36.8% | 30.9% | 18.4% |
| Sustained pain free** | 22.3% | 15.1% | 9.4% |

*pain reduction from moderate or severe to mild or none
**no recurrence of pain and no rescue within 24 hours

### Example 3

### Efficacy of Diclofenac Powder Against Primary and Secondary Migraine Endpoints

A phase III clinical trial was undertaken in adult migraine patients. 690 migraine patients were randomized among treatments and a comparison made among treatments with a 50 mg. diclofenac potassium sachet formulation prepared substantially as described in Example 1, and demonstrating a tₘₐₓ of about 14 minutes, and placebo. The efficacy of the treatment against four primary endpoints (headache pain, nausea, photophobia and phonophobia) are reported in Table 6.

TABLE 6

| | PRO-513 | Placebo |
|---|---|---|
| Headache Pain^{a} | | |
| Number of Subjects | 343 | 347 |
| No Pain | 86 (25.1%) | 35 (10.1%) |
| Mild, Moderate, or Severe Pain | 257 (74.9%) | 312 (89.9%) |

| | |
|---|---|
| P-Value^{b} | <0.001 |

| Nausea^{a} | | |
|---|---|---|
| Number of Subjects | 343 | 347 |
| No Nausea | 222 (64.7%) | 183 (52.7%) |
| Mild, Moderate, or Severe Nausea | 121 (35.3%) | 164 (47.3%) |
| P-Value^{b} | 0.002 | |
| Photophobia^{a} | | |
| Number of Subjects | 343 | 347 |
| No Photophobia | 139 (40.5%) | 95 (27.4%) |
| Mild, Mod., or Sev. Photophobia | 204 (59.5%) | 252 (72.6%) |

| | |
|---|---|
| P-Value^{b} | <0.001 |

| | | |
|---|---|---|
| Phonophobia^{a} | | |
| Number of Subjects | 343 | 347 |
| No Photophobia | 152 (44.3%) | 95 (27.4%) |
| Mild, Mod or Sev Photophobia | 191 (55.7%) | 252 (72.6%) |

| | |
|---|---|
| P-Value^{b} | <0.001 |

^{a}Based on Assessments at 2 Hours Post Dose
^{b}P-Value from a Cochran-Mantel-Haenszel test, stratified by analysis center.

A graphical summary of headache intensities during the first twenty four hours after treatment, comparing a fast release diclofenac sachet formulation and placebo, is reported in FIG. 2. Sustained pain free and recurrence rates are reported below in Table 7:

TABLE 7
Sustained Pain Free Response Rates

| Sustained PF | PRO-513 (N = 343) | Placebo (N = 347) | P-Value^{a} |
|---|---|---|---|
| Yes | 65 (19.0%) | 25 (7.2%) | <0.001 |
| No | 278 (81.0%) | 322 (92.8%) | |

Recurrence Rates

| Recurrence | PRO-513 (N = 86) | Placebo (N = 35) |
|---|---|---|
| Yes | 21 (24.4%) | 10 (28.6%) |
| No | 65 (75.6%) | 25 (71.4%) |

Time to Recurrence

| | Median Time (Hours) | 95% C.I |
|---|---|---|
| PRO-513 | >24 | (24.0 to >24) |
| Placebo | >24 | (24.0 to >24) |

^{a}P-Value from a Cochran-Mantel-Haenszel test stratified by analysis center

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1) A process for treating pain in a mammal which comprises administering to the mammal an amount of a pharmaceutical composition effective to provide an analgesic effect, said pharmaceutical composition comprising diclofenac or a pharmaceutically acceptable salt thereof and an opioid selected from hydrocodone, oxycodone, tramadol or fentanyl, or a pharmaceutically acceptable salt thereof, wherein:
a) greater than 85% of said diclofenac or diclofenac salt is released from said formulation in less than twenty minutes when tested in water having a pH of 6.8 at 37° C. at 50 RPM using a USP type II dissolution apparatus;
b) greater than 85% of said opioid is released from said formulation in less than sixty minutes when tested at 37° C.:
i) in a USP type I basket at 100 rpm in 0.1N HCl (tramadol);
ii) in a USP type I basket at 100 rpm in a phosphate buffered pH 7.2 aqueous medium (oxycodone);
iii) in a USP type II paddle device at 50 rpm in a phosphate buffered pH 7.2 aqueous medium (hydrocodone); or
iv) in water buffered by 0.1M HCl at 37° C. at 50 RPM using a USP Type II dissolution apparatus (fentanyl); and
c) the weight ratio of diclofenac to opioid is within a range that the administration of a therapeutic amount of said composition to a mammal will provide a greater analgesic effect than the effect obtainable by use of either said diclofenac or said opioid alone.

2) The process of claim 1 wherein greater than 85% of said diclofenac or diclofenac salt is released from said formulation in less than ten minutes when tested as described in claim 1.

3) The process of claim 1 wherein less than 35% of said opioid is released from said formulation in less than ten minutes when tested as described in claim 1.

4) The process of claim 1 wherein:
a) greater than 85% of said diclofenac or diclofenac salt is released from said formulation in less than ten minutes when tested as described in claim 1; and
b) less than 35% of said opioid is released from said formulation in less than ten minutes when tested as described in claim 1.

5) The process of claim 1 wherein said process comprises administering said composition three times daily.

6) The process of claim 1 wherein:
a) said composition provides eight hours or more of relief from acute pain; and
b) said composition excludes time means for extending or delaying the release of active ingredient from said composition.

7) The process of claim 1 which comprises administering to said mammal a dosage unit comprising from about 12.5 to about 50 mg. of diclofenac or a pharmaceutically acceptable salt thereof and from about 2.5 to about 10.0 mg. of hydrocodone or a pharmaceutically acceptable salt thereof.

8) The process of claim 1 which comprises administering to said mammal a dosage unit comprising from about 12.5 to about 50 mg. of diclofenac or a pharmaceutically acceptable salt thereof and from about 2.5 to about 10.0 mg. of oxycodone or a pharmaceutically acceptable salt thereof.

9) The process of claim 1 which comprises administering to said mammal a dosage unit comprising from about 12.5 to about 50 mg. of diclofenac or a pharmaceutically acceptable salt thereof and from about 2.5 to about 10.0 mg. of tramadol or a pharmaceutically acceptable salt thereof.

10) The process of claim 1 which comprises administering to said mammal a dosage unit comprising from about 12.5 to about 50 mg. of diclofenac or a pharmaceutically acceptable salt thereof and from about 0.2 to about 1.6 mg. of fentanyl or a pharmaceutically acceptable salt thereof.

11) A fixed combination dosage form comprising:
a) diclofenac or a pharmaceutically acceptable salt thereof, wherein greater than 85% of said diclofenac or diclofenac salt is released from said formulation in less than twenty minutes when tested in water having a pH of 6.8 at 37° C. at 50 RPM using a USP type II dissolution apparatus; and
b) greater than 85% of said opioid is released from said formulation in less than sixty minutes when tested at 37° C.:
i) in a USP type I basket at 100 rpm in 0.1N HCl (tramadol);
ii) in a USP type I basket at 100 rpm in a phosphate buffered pH 7.2 aqueous medium (oxycodone);
iii) in a USP type II paddle device at 50 rpm in a phosphate buffered pH 7.2 aqueous medium (hydrocodone); or
iv) in water buffered by 0.1M HCl at 37° C. at 50 RPM using a USP Type II dissolution apparatus (fentanyl).

12) The dosage form of claim 11 wherein greater than 85% of said diclofenac or diclofenac salt is released from said formulation in less than ten minutes when tested as described in claim 1.

13) The dosage form of claim 11 wherein less than 35% of said opioid is released from said formulation in less than ten minutes when tested as described in claim 1.

14) The dosage form of claim 11 wherein:
a) greater than 85% of said diclofenac or diclofenac salt is released from said formulation in less than ten minutes when tested as described in claim 1 and
b) less than 35% of said opioid is released from said formulation in less than ten minutes when tested as described in claim 1.

15) The dosage form of claim 11 wherein:
a) said composition provides eight hours or more of relief from acute pain; and
b) said composition excludes means for extending or delaying the release of active ingredient from said composition.

16) The dosage form of claim 11 which comprises:
a) from about 12.5 to about 50 mg. of diclofenac or a pharmaceutically acceptable salt thereof, and
b) from about 2.5 to 10.0 mg. of hydrocodone or a pharmaceutically acceptable salt thereof.

17) The dosage form of claim 11 which comprises:
a) from about 12.5 to about 50 mg. of diclofenac or a pharmaceutically acceptable salt thereof, and
b) from about 2.5 to 10.0 mg. of oxycodone or a pharmaceutically acceptable salt thereof.

18) The dosage form of claim 11 which comprises:
a) from about 12.5 to about 50 mg. of diclofenac or a pharmaceutically acceptable salt thereof, and
b) from about 25.0 to 50.0 mg. of tramadol or a pharmaceutically acceptable salt thereof.

19) The dosage form of claim 11 which comprises:
a) from about 12.5 to about 50 mg. of diclofenac or a pharmaceutically acceptable salt thereof, and
b) from about 0.2 to 1.6 mg. of fentanyl or a pharmaceutically acceptable salt thereof.

20) A process for treating pain in a mammal which comprises administering to the mammal an amount of a pharmaceutical composition effective to provide an analgesic effect, said pharmaceutical composition comprising diclofenac or a pharmaceutically acceptable salt thereof and an opioid selected from hydrocodone, oxycodone, tramadol or fentanyl, or a pharmaceutically acceptable salt thereof, wherein:
a) said composition exhibits a tₘₐₓ for said diclofenac or diclofenac salt of from about 10 minutes to about 30 minutes; and
b) the weight ratio of diclofenac to opioid is within a range that the administration of a therapeutic amount of said composition to a mammal will provide a greater analgesic effect than the effect obtainable by use of either said diclofenac or said opioid alone.

21) The process of claim 20 which comprises administering to said mammal a dosage unit comprising (i) from about 12.5 to about 50 mg. of diclofenac or a pharmaceutically acceptable salt thereof and (ii) from about 2.5 to about 10.0 mg. of hydrocodone or a pharmaceutically acceptable salt thereof, from about 2.5 to about 10.0 mg. of oxycodone or a pharmaceutically acceptable salt thereof, from about 2.5 to about 10.0 mg. of tramadol or a pharmaceutically acceptable salt thereof, or from about 0.2 to about 1.6 mg. of fentanyl or a pharmaceutically acceptable salt thereof.

22) A pharmaceutical composition which comprises diclofenac or a pharmaceutically acceptable salt thereof and an opioid selected from hydrocodone, oxycodone, tramadol or fentanyl, or a pharmaceutically acceptable salt thereof, wherein:
a) said composition exhibits a tₘₐₓ for said diclofenac of from about 10 minutes to about 30 minutes; and
b) the weight ratio of diclofenac to opioid is within a range that the administration of a therapeutic amount of said composition to a mammal will provide a greater analgesic effect than the effect obtainable by use of either said diclofenac or said opioid alone.

23) The composition of claim 22 which comprises administering to said mammal a dosage unit comprising (i) from about 12.5 to about 50 mg. of diclofenac or a pharmaceutically acceptable salt thereof and (ii) from about 2.5 to about 10.0 mg. of hydrocodone or a pharmaceutically acceptable salt thereof, from about 2.5 to about 10.0 mg. of oxycodone or a pharmaceutically acceptable salt thereof, from about 2.5 to about 10.0 mg. of tramadol or a pharmaceutically acceptable salt thereof, or from about 0.2 to about 1.6 mg. of fentanyl or a pharmaceutically acceptable salt thereof.
